(19) United States
(12) Patent Application Publication
(10) Pub. No.: US 2004/0057924 A1
(43) Pub. Date: Mar. 25, 2004

(54) Holistic dermatological cleanser

(76) Inventor: Cristina L. Gustavsson, Everett, MA (US)

Correspondence Address: DAVIS & BUJOLD, P.L.L.C., FOURTH FLOOR, 500 N. COMMERCIAL STREET, MANCHESTER, NH, 03101-1151, US

(21) Appl. No.: 10/252,991

(22) Filed: Sep. 23, 2002

(51) Int. Cl. A61K 35/78; A61K 7/06; A61K 35/20

(52) U.S. Cl. 424/74; 424/744; 424/535; 424/745; 424/750; 424/766; 424/617

(57) ABSTRACT

A natural skin lotion, salve or conditioner made from wholesome, naturally grown organic plants for reconditioning and regulating skin conditions and the use of such a natural, healthful skin lotion with the benefits of alternative healing elements, namely crystal therapy to produce benefits which can reach beyond the physical, and to emotional well being.

### FIELD OF THE INVENTION

The present invention relates to the field of skin care, in particular to healthful care of facial skin using an all natural dermatological lotion, ointment or salve and in combination with crystal therapy and holistic medicinal treatment and, more specifically to infusing the lotion, ointment or salve with a programmed crystalline energy from a particular crystal either accommodated in the ointment or salve or ointment and salve having been exposed for a time period to the crystal.

### BACKGROUND OF THE INVENTION

Many personal care products currently available to consumers are directed primarily to improving the health and/or physical appearance of the skin. Among these skin care products, many are directed to delaying, minimizing or even eliminating skin wrinkling and other histological changes typically associated with the aging of skin or environmental damage to human skin.

Skin is subject to damage from many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin and also from emotional and metaphysical changes in an individual. Whether extrinsic or intrinsic, these factors result in visible signs of skin aging and environmental damage, such as wrinkling and other forms of roughness (including increased pore size, flaking and skin lines), and other histological changes associated with skin aging or damage. To many people, skin wrinkles are a reminder of the disappearance of youth. As a result, the elimination of wrinkles has become a booming business in youth-conscious societies. Treatments range from cosmetic creams and moisturizers to various forms of cosmetic surgery.

Holistic and alternative medicine utilize the effects of crystals and their energies ability to aid in the healing and therapeutic process. Crystals are imbued with what is often referred to in the art as vibrational medicine predicated upon the fact that crystals, for example, quartz, vibrate at particular frequencies and can play a role in therapeutic or medicinal effects on the body which can result in both physical benefits as well as emotional benefits and are also believed to provide a balancing of mind and body relationship.

Crystals are generally composed of silica and oxygen in a crystalline matrix and are a medium through which personal energies can be amplified. Quartz, which constitutes a high percentage of the Earth's crust is often used however, other precious and semi-precious stones and metals which occur in lower quantities may also be used in light of their crystalline qualities. Quartz crystals in particular are desirable because they carry the energies of higher light frequencies which can be transformational when used in holistic and alternative medicines.

The crystalline and tetrahedral shape of crystals assist in amplifying a persons energy as it is transferred through the terminations of the crystal. Crystals work best with white light and have one of the highest frequencies capacities in the entire mineral kingdom. Quartz crystals often have the most versatile spectrum range in the group as it not only channels white light but, in view of the fact that white light is composed of all the colors in the spectrum, quartz crystals can correspondingly channel the other colors as well.

Everything in the world is to some extent crystalline, and when crystals resonate in precisely programmed frequencies they are able to work with, and be effected by and transmit a multitude of different energies. In particular, when used in the human body, which is theoretically crystalline in nature, these energies may beneficially effect organs, including the body's largest organ, the derma or skin, and the blood system in a manner that western medicine does not. In order to assist the reader in understanding the present description, the following definitions are provided.

Allopathic medicine: mainstream conventional western medical practices.

Alternative healing practice: a healing practice other than conventional western (allopathic) approaches.

Alternative medicine: healing practices other than conventional western (allopathic) approaches.

Alternative healing elements: physical objects used in connection with various alternative healing practices.

Aroma therapy: an ancient healing art in which essential oils from herbs and flowers are applied to the skin or inhaled to address physical, emotional and spiritual imbalances. Essential oils of herbs, flower, plants, trees and spices are also often used to create an energy balance.

Holistic: healing practices that consider the mind, body and spirit as a whole and appropriately integrate many traditions of healing as they apply to the individual person and situations.

Crystals: crystals, gemstones, glass beads, minerals, metals and stones which are prized for their beauty color, appearance and shape and which can be programmed accordingly to focus a particular desired energy and frequency into a desired object or environment.

Energy: subtle electrical and metaphysical energies which interact with the energy system of the body to stabilize emotional, physical, mental and spiritual states and assist in the wellness and healing of both physical, emotional and metaphysical levels of the consciousness and unconsciousness of the body.

Waters: not meant to convey normal H₂O, but used herein to refer generally the mixture of distilled water and other substantially similar viscous fluids

While not formally recognized according to the standards of allopathic medicine as having healing powers, there are a number of alternative practices for promoting health and well being which trace their roots deep into human history. More and more people are turning to these ancient practices and more and more investigations are discovering their power. The effectiveness of these holistic healing practices said to be enhanced by and in some cases dependent upon receiving their powers with an open heart and a feeling of love and affection.

### SUMMARY OF THE INVENTION

The present invention utilizes a natural skin lotion, salve or conditioner made from wholesome, naturally grown organic plants which provides particularly excellent results in reconditioning and regulating skin conditions. In addition, the combination of the use of such a natural, healthful skin lotion with the benefits of alternative healing elements, namely crystal therapy can produce benefits which reach beyond the physical, and to emotional well being.

Wherefore, it is an object of the present invention to overcome the above mentioned shortcomings and drawbacks associated with the prior art.

An object of the present invention is to provide an holistic dermatological ointment which when used in combination with alternative healing elements is infused with vibrational energy to promote emotional and physical well being as well as cleansing and care of the derma.

A further object of the present invention is to provide a holistic dermatological ointment or salve substance infused with vibrational energy from a crystal which is in constant contact with the ointment or salve within a package or container.

Yet another object of the present invention is to provide a holistic dermatological ointment or salve without a crystal element in the substance or container, but which has been infused with metaphysical energy from a crystal exposed for a period of time to the ointment or salve within a package or container.

A still further object of the present invention is to provide a method of infusing a dermatological ointment or salve with metaphysical energy from a crystal.

The present invention relates to a skin care composition comprising a waters mixture comprising; distilled water and aloe vera mixed in a ratio of about 2:1; vegetable glycerin is added to the distilled water and aloe vera mixture in a ratio of about 1:8; and at least one of an herb and flower chosen from a group of aromatic therapeutic herbs and flowers; an oils mixture comprising a natural plant extract oil is mixed with approximately equal amounts of kokum butter and shea butter in a ratio of about 3:1; a desired amount of cocoa butter; and an amount of emulsifying wax in a range of about 1:8; the oils mixture being combined at a temperature of about 110°-150° F., blended to a slurry and cooled to room temperature wherein the waters mixture is combined with the oils mixture and blended to produce the skin care composition.

The present invention also relates to an all natural skin care composition infused with healing energy comprising a waters mixture comprising distilled water and aloe vera, vegetable glycerin, and at least one of an herb and flower chosen from a group of aromatic therapeutic herbs and flowers; an oils mixture comprising a natural plant extract oil mixed with kokum butter, shea butter and a desired amount of cocoa butter and a desired amount of emulsifying wax at a temperature of about 110°-150° F., blended to a slurry and cooled to room temperature; a holistic crystal is provided, the crystal having a desired program for facilitating at least one of the physical and emotional balance and healing of the user; and wherein the waters mixture is combined with the oils mixture and blended to produce the skin care composition to which is added the programmed holistic crystal to infuse the skin care composition with healing vibrational energy.

The present invention also relates to a method of making an all natural skin care lotion comprising the steps of formulating a waters mixture by the steps of;

mixing distilled water and aloe vera in a ratio of about 2:1; adding vegetable glycerin to the distilled water and aloe vera mixture in a ratio of about 1:8; and adding at least one of an herb and flower chosen from a group of aromatic therapeutic herbs and flowers; formulating an oils mixture by the steps of; mixing a natural plant extract oil with approximately equal amounts of kokum butter and shea butter in a ratio of about 3:1; adding a desired amount of cocoa butter; and adding an amount of emulsifying wax in a range of about 1:8; combining the oils mixture at a temperature of about 110°-150° F. and blending the combined oils mixture to a slurry and cooling the slurry to room temperature; combining the waters mixture with the oils mixture and mixing to produce the skin care lotion; and infusing the skin care lotion with holistic energy from a holistic crystal.

### BRIEF DESCRIPTION OF THE DRAWINGS

The invention will now be described, by way of example, with reference to the accompanying drawings in which:

FIG. 1 is a flow diagram depicting the general procedure for the present invention;

FIG. 2 is shows the steps and method of combining the ingredients for the skin care lotion;

FIG. 3 details the steps of combining the lotion with a programmed crystal; and

FIG. 4 shows an embodiment of the finished product for use and sale.

### DETAILED DESCRIPTION OF THE INVENTION

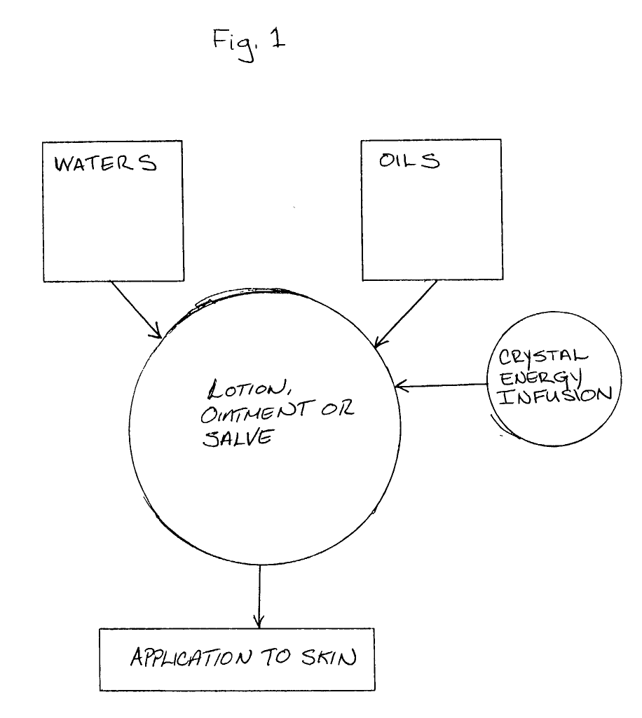

FIG. 1, depicts in general the method described below which combines a waters mixture with chosen oils to form a healthful skin lotion substance which is imbued with a desired crystal induced vibrational energy and finally applied to a user's skin. The term “waters” relates to more than conventional H₂O, and includes the overall initial mixture including at least the distilled waters, aloe vera, vegetable glycerine, herbs and flowers discussed below.

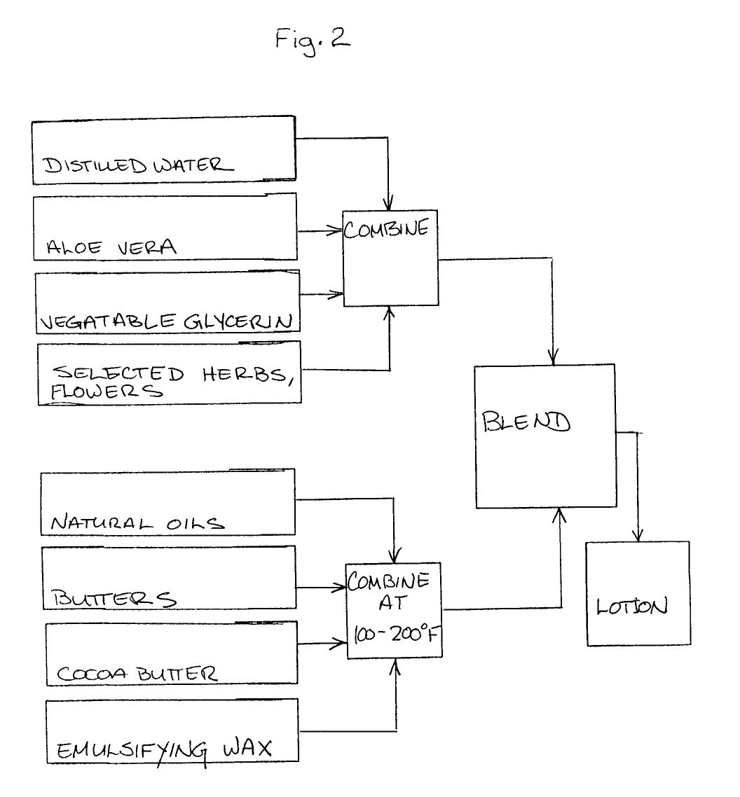

Turning to FIG. 2, first, the waters are prepared by mixing a desired unit of distilled water with aloe vera in an amount of approximately 2:1 ratio. To this is added a desired amount of vegetable glycerine in the range of about 0.1:1 ratio (0.5 to 5 ounces per cup of distilled water and aloe vera) along with desired herbs and flowers from the aromatherapy field, for example, calendula, combre and lavender. A further discussion of these herbs and flowers and their relative properties is provided below.

Secondly, an oil mixture is prepared wherein a particular amount of oil from either wheat germ, grape seed or jojobat sweet almond is mixed with butters in a ratio of approximately 3:1. The butters are preferably kokum butter or shea butter or a mixture of the two. To the oil and butter mixture is added a small desired amount of cocoa butter along with a similarly small amount of emulsifying wax or vegetable emulsifying wax to complete the oil mixture. The oil mixture is melted at a temperature of about 100° F. to 200° F. and more preferably 110° F. to 150° F. for a time period of about five (5) to fifteen (15) minutes. The oils are poured into a blender and allowed to cool at room temperature. During the cooling process, the oils may be blended, i.e., mixed in the blender, until a thick and creamy blend of materials is produced.

The waters are now added to the oil mixture in a ratio of about 1:1, although some deviations therefrom may be obviously undertaken to achieve a desired consistency. With the oil mixture continuing to be mixed in the blender, the waters are slowly poured into the center of the vortex formed by the whirling oil mixture. Once the waters are completely mixed with the oils in the blender, the blender is set at its highest speed until the mixture is brought to a semi-solid consistency.

When the preparation of the ointment or salve is complete, a stone or crystal is chosen which has been programmed with a desired energy to be infused into the ointment. The chosen stone or crystal or plurality of stones or crystals can be mixed and matched, for instance, to infuse various energies and frequencies into the ointment. A further discussion of programming stones or crystals is provided below. The stones or crystal and/or a mixture thereof are placed into a container to which the completed ointment is added. The container is generally of a volume which can be sold at the retail level to individual consumers for example in the range of 1 to 20 ounces and more preferably in the range of 4 to 10 ounces of ointment or salve. The container is sealed and the stones and/or crystals infuse their programmed energies into the ointment for a certain period of time being at least 24 hours or more, and most preferably 60 hours or more.

In some cases the stone may be removed from the ointment, for instance for reprogramming, reuse, etc before the product is sold or used. Otherwise the stones and crystals may be left in the ointment to continue to release their energies and frequencies therein continuously, and may be used after the ointment or salve has been used up. Once a desired minimum infusion time is complete, i.e., at least 24 hours, the energized ointment or salve is complete and can be applied to the skin of all types of people of all ages and provides healing power beyond the physical level but also to the any of the metaphysical levels, i.e., emotional.

Any number of stones or crystals may be used for their particular desirable energies and frequencies. These are as follows, but not limited specifically to; rose quartz, for love and healing old wounds; morganite for the same purpose; amethyst, and angelite which aids in relieving water retention, swelling and permits communication with angels at the metaphysical level; blue lace agate has a calming effect and can help restore blood flow to the capillaries and relieve arthritis; aquamarine which aids in communication and public speaking abilities; florite which assists in detoxification, enhancing the memory and strengthening bones can also be used.

Different crystal types may also be used, the most common being: double terminated crystals; record keeper crystals; elestial crystals; channeling crystals; tabular crystals; phantom crystals; self healed crystals; window crystals; laser crystals. The most preferable crystal to use with the above discussed salve or ointment being rose quartz which is used mainly due to its particular properties relating to beauty and love.

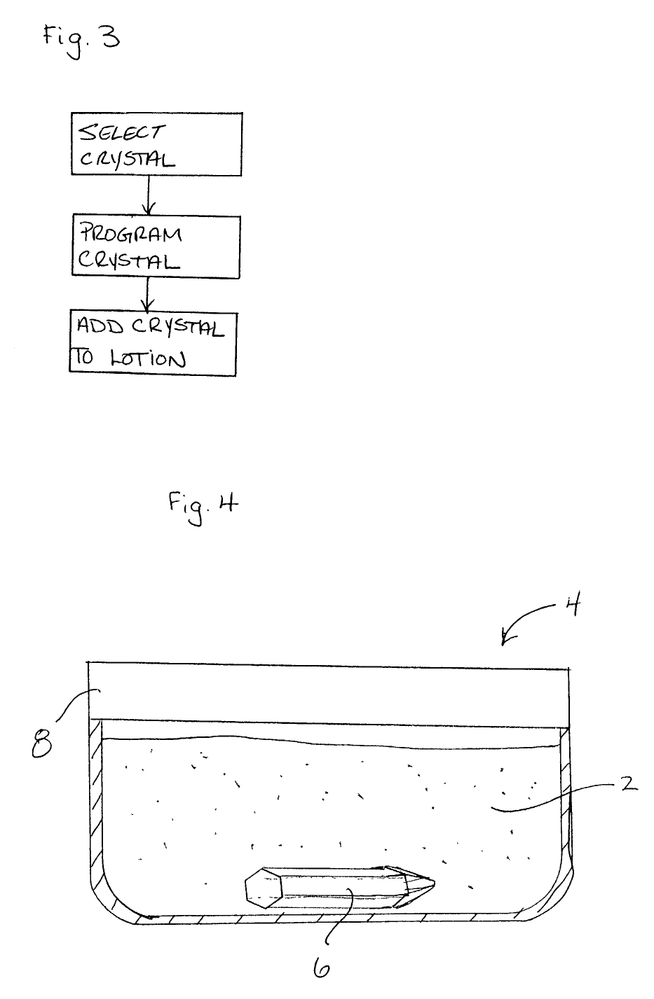

Turning to FIG. 3, a selected crystal, or crystals, utilized in the skin care lotions may also be programmed to facilitate healing or achieve a desired healthful result in a user of the crystal-or in the present case, of the lotion. Crystal programming may be accomplished by a number of different methods, some of which are known as manifestation and intention. The method of programming discussed herein is based on the programmers intent. The intent of the programmer is passed to the crystal to focus the vibrational energy of the crystal based on the programmers intent. Shamballa, a type of energy healing may be used for this purpose. A program is selected for a crystal based upon the needs of a user of the skin care lotion in order to facilitate a desired result. For example programs, imparted by the known methods of Shamballa, may be imparted to a crystal to heal eczema, rosaceae and swelling of joints. It should also be noted that other programs relating to any number of afflictions could also be utilized, for instance programs may be used to facilitate a user overcoming depression, apathy, anxiety or any other type of physical or mental affliction can be used. In other words, the programs need not be directly related to skin conditions per se in all cases.

Initially, the crystal is de-programmed, or cleared of unwanted energies. This can be done in a number of ways, one method being directly contacting the crystal with both hands, usually holding the base of the crystal in one hand (usually the right hand) and the point in the other, a connection is made with the crystal using Cho Ku Rei, using the Sei He Ki symbol to clear the crystal of accumulated energies followed by Cho Ku Rei again. De-programming may need to be affirmed by repeating the process, often up to three (3) times to ensure that all accumlated energies are de-programmed.

Once the crystal is de-programmed, the crystal is ready to be imbued with a new program or energy or a number of programs or energies. Once the programmer has discerned the needs of the user, a desired program to address these needs can be imparted to the crystal. A similar method of holding the crystal and imparting a desired intent to the crystal as described above with respect to de-programming can be used to program the crystal. Once the crystal has been provided with a desired healing energy, it can then be introduced into the lotion, ointment or salve to facilitate both physical and emotional healing of the user.

Selection of particular herbs and flowers can also be important in providing the lotion, ointment or salve with a particularly unique or pleasing aroma. Calendula is flower, comfrey is prepared from a plant utilized for cell regeneration, and lavender is derived from an herb.

Once all the above discussed necessary steps have been accomplished, as seen in FIG. 4, the blended lotion 2 may be added to a container 4 having a chosen and programmed crystal 6 as discussed above. The crystal 6 is confined within the container 4 being sealed for instance by a lid or cover 8 to allow the vibrational energy to be absorbed by the lotion 2. After the at least 24 hour period, the crystal 6 may be removed from the container 4 and the lotion 2, or it may remain in to provide further vibrational energy or to be kept by the user following use of all the lotion.

In view of the above description a specific recipe is set forth for producing approximately 8-10 ounces of skin lotion:

Waters:

⅔ cup distilled water

⅓ cup aloe vera

1 oz. vegetable glycerine

1 drop each of calendula, comfrey and lavender oils;

Oils:

¾ cup oil (wheat germ, grapeseed, jojobat sweet almond)

⅓ cup kokum butter and shea butter;

1 Tsp cocoa butter

1 oz. emulsifying wax (vegetable)

A second embodiment of the present invention relates to a dry skin cleanser which may also be produced by combining:

6 ounces of aloe vera;

1 ounce of vegetable glycerine;

¼ tsp. shea butter;

3 Tbsp. butter;

2 Tbsp. cornmeal;

3 drops calendula oil;

1 drop lavender oil;

2 drops comfrey oil;

Another embodiment for treatment of oily skin:

6 oz. Aloe vera;

3 Tbsp. sea salt;

1 oz. vegetable glycerine;

3 Tbsp. cornmeal;

3 drops calendula oil;

1 drop of lavender oil;

2 drops comfrey oil;

A further embodiment for a masque for all skin types:

8 oz. aloe vera gel;

¼ tsp. chamomile;

3 drops calendula oil;

2 drops comfrey oil;

1 drop lavender oil;

In a still further embodiment for a skin toner:

6 oz distilled water;

1 oz. chamomile;

1 oz. green tea;

3 drops calendula oil;

1 drop rose oil;

Since certain changes may be made in the above described improved dermatological treatment lotions without departing from the spirit and scope of the invention herein involved, it is intended that all of the subject matter of the above description or shown in the accompanying drawings shall be interpreted merely as examples illustrating the inventive concept herein and shall not be construed as limiting the invention.

Wherefore, I/We claim:

1. A skin care composition comprising:
a waters mixture comprising;
distilled water and aloe vera mixed in a ratio of about 2:1;
vegetable glycerin is added to the distilled water and aloe vera mixture in a ratio of about 1:8; and
at least one of an herb and flower chosen from a group of aromatic therapeutic herbs and flowers;
an oils mixture comprising
a natural plant extract oil is mixed with approximately equal amounts of kokum butter and shea butter in a ratio of about 3:1;
a desired amount of cocoa butter; and
an amount of emulsifying wax in a range of about 1:8;
the oils mixture being combined at a temperature of about 110°-150° F., blended to a slurry and cooled to room temperature wherein the waters mixture is combined with the oils mixture and blended to produce the skin care composition.

2. The skin care composition as set forth in claim 1, wherein the at least one of an herb and flower is chosen from the group of aromatic therapeutic herbs and flowers comprising, calendula, comfrey and lavender.

3. The skin care composition as set forth in claim 2, wherein the natural plant extract oil is chosen from the group of: wheat germ oil, grape seed oil, jojobat sweet almond oil.

4. The skin care composition as set forth in claim 3 further comprising a holistic crystal being added to the skin care composition for infusing the skin care composition with energy from the crystal.

5. The skin care composition as set forth in claim 4, wherein the holistic crystal is added to the skin care composition for a time period of at least 24 hours to facilitate the infusion of holistic energy into the skin care composition.

6. An all natural skin care composition infused with healing energy comprising:
a waters mixture comprising distilled water and aloe vera, vegetable glycerin, and at least one of an herb and flower chosen from a group of aromatic therapeutic herbs and flowers;
an oils mixture comprising a natural plant extract oil mixed with kokum butter, shea butter and a desired amount of cocoa butter and a desired amount of emulsifying wax at a temperature of about 110°-150° F., blended to a slurry and cooled to room temperature;
a holistic crystal is provided, the crystal having a desired program for facilitating at least one of the physical and emotional balance and healing of the user; and
wherein the waters mixture is combined with the oils mixture and blended to produce the skin care composition to which is added the programmed holistic crystal to infuse the skin care composition with healing vibrational energy.

7. The all natural skin care composition infused with healing energy as set forth in claim 6, wherein the distilled water and aloe vera are mixed in a ratio of about 2:1, and the vegetable glycerin is added to the distilled water and aloe vera mixture in a ratio of about 1:8, and in the oils mixture the natural plant extract oil is mixed with approximately equal amounts of the kokum butter and shea butter in a ratio of about 3:1, and the amount of emulsifying wax added is in a range of about 1:8.

8. The all natural skin care composition infused with healing energy as set forth in claim 7, wherein the at least one of an herb and flower is chosen from the group of aromatic therapeutic herbs and flowers comprising, calendula, comfrey and lavander.

9. The all natural skin care composition infused with healing energy as set forth in claim 8, wherein the natural plant extract oil is chosen from the group of: wheat germ oil, grape seed oil, jojobat sweet almond oil.

10. A method of making an all natural skin care lotion comprising the steps of:
formulating a waters mixture by the steps of;
mixing distilled water and aloe vera in a ratio of about 2:1;
adding vegetable glycerin to the distilled water and aloe vera mixture in a ratio of about 1:8; and
adding at least one of an herb and flower chosen from a group of aromatic therapeutic herbs and flowers;
formulating an oils mixture by the steps of;
mixing a natural plant extract oil with approximately equal amounts of kokum butter and shea butter in a ratio of about 3:1;
adding a desired amount of cocoa butter; and
adding an amount of emulsifying wax in a range of about 1:8;
combining the oils mixture at a temperature of about 110°-150° F. and blending the combined oils mixture to a slurry and cooling the slurry to room temperature;
combining the waters mixture with the oils mixture and mixing to produce the skin care lotion; and
infusing the skin care lotion with holistic energy from a holistic crystal.

11. The method of making an all natural skin care lotion as set forth in claim 10 further comprising the step of choosing the at least one of the herb and flower from the group of aromatic therapeutic herbs and flowers comprising, calendula, comfrey and lavander.

12. The method of making an all natural skin care lotion as set forth in claim 11 further comprising the step of choosing the natural plant extract oil from the group of: wheat germ oil, grape seed oil, jojobat sweet almond oil.

13. The method of making an all natural skin care lotion as set forth in claim 10 further comprising the step of placing the holistic crystal in direct contact with the skin care lotion for a desired time period.

14. The method of making an all natural skin care lotion as set forth in claim 10 further comprising the step of placing the holistic crystal in direct contact with the skin care lotion for a time period of no less then 24 hours and subsequently removing the holistic crystal from contact with the skin care lotion.

15. The method of making an all natural skin care lotion as set forth in claim 10 further comprising the step of programming the crystal with a desired healing energy directed to facilitating emotional and physical balance and healing of a user of the skin care lotion.
